(19) United States
(12) Patent Application Publication
(10) Pub. No.: US 2026/0062377 A1
(43) Pub. Date: Mar. 5, 2026

(54) IMPROVED PROCESS FOR PREPARING P-AMINOBENZOIC ACID-2-ETHYLHEXYL ESTER

(71) Applicant: BASF SE, Ludwigshafen am Rhein (DE)

(72) Inventors: Peter LEIDINGER, Ludwigshafen am Rhein (DE); Thomas BERG, Ludwigshafen am Rhein (DE); Helmut KRONEMAYER, Ludwigshafen am Rhein (DE); Sebastian EMMERLING, Inchera (IE); Joerg WERLE, Ludwigshafen am Rhein (DE)

(21) Appl. No.: 19/106,752

(22) PCT Filed: Aug. 28, 2023
(86) PCT No.: PCT/EP2023/073497

§ 371 Date: Feb. 26, 2025

(30) Foreign Application Priority Data
Aug. 29, 2022 (EP) 22192662.9

(51) Int. Cl. C07C 227/04; B01J 23/46; C07C 201/12; C07C 227/40; C07C 227/44; C07C 229/60; C07D 251/70

(52) U.S. Cl. CPC C07C 227/04; B01J 23/464; C07C 201/12; C07C 227/40; C07C 227/44; C07C 229/60; C07D 251/70; C07C 2523/42; C07C 2523/44; C07C 2523/46; C07C 2525/02

(57) ABSTRACT

The present invention relates to a process for preparing p-aminobenzoic acid-2-ethylhexyl ester comprising reacting p-nitrobenzoic acid-2-ethylhexyl ester with hydrogen in the presence of a catalyst, wherein the reaction is performed in water or in a mixture of water and 2-ethylhexanol. Further, the present invention relates to the p-aminobenzoic acid-2-ethyl hexylester obtained by the process and having high purity.

The present invention relates to a process for preparing p-aminobenzoic acid-2-ethylhexyl ester comprising reacting p-nitrobenzoic acid-2-ethylhexyl ester with hydrogen in the presence of a catalyst, wherein the reaction is performed in water or in a mixture of water and 2-ethylhexanol. Further, the present invention relates to the p-aminobenzoic acid-2-ethyl hexylester obtained by the process and having high purity.

Ethylhexyl triazone (Uvinul T 150, CAS number: 88122-99-0) is a known highly effective UVB filter. Due to its beneficial physical and chemical properties, it is widely used as ingredient in various cosmetic preparations such as anti-aging face care products and sunscreens. The exceptional photo-stability and absorptivity of ethylhexyl triazone leads to the requirement of only small concentrations to achieve a high sun protection factor. Further, the polarity of the structure provides good solubility in cosmetic oils as well as high affinity to the skin's keratin.

Therefore, an economically and environmentally optimal industrial process for the preparation of ethylhexyl triazone is highly desirable. Considering the contact with human skin in its application in cosmetic preparations, ethylhexyl triazone is required in high purity. Typically, ethylhexyl triazone is prepared in a 3-step process starting with the esterification of p-nitrobenzoic acid. The formed p-nitrobenzoic acid-2-ethylhexyl ester is then reduced to the p-aminobenzoic acid-2-ethylhexyl ester, which subsequently reacts with a cyanuric halide to the final product.

As regards the second step, i.e., the reduction of p-nitrobenzoic acid-2-ethylhexyl ester to p-aminobenzoic acid-2-ethylhexyl ester, EP 3 674 293 A1 discloses the reduction of p-nitrobenzoic acid esters either by catalytic hydrogenation in a mixture of ethanol and methanol or using a catalyst and a reducing agent in an aprotic organic solvent. Further, it is considered that protic solvents are disadvantageous because these solvents, especially water, may give rise to unwanted side reactions in the subsequent reaction to ethylhexyl triazone. The resulting side products would need to be removed which requires additional purification steps.

CN112321522 discloses a process for the preparation of p-aminobenzoic acid-2-ethylhexyl ester, wherein isooctyl p-aminobenzoate is reduced by catalytic hydrogenation in isooctyl alcohol.

Considering the high requirements on the purity of ethylhexyl triazone, also its starting material, p-amino benzoic acid-2-ethyl hexylester, is required in optimum product quality. Therefore, it was an object of the present invention to provide an improved process for the preparation of p-aminobenzoic acid-2-ethylhexyl ester with optimum product quality.

In this regard, it was an object to provide a process which ensures high product purity. Further, it was an object to provide a process which is economically and environmentally advantageous. Further, it was an object to provide a process, which is technically and economically suitable for large-scale industrial applications.

It has surprisingly been found that at least some of the above objects can be achieved by the subject matter of the present invention as described hereinafter and in the claims.

In one embodiment, the present invention relates to a process for preparing p-aminobenzoic acid-2-ethylhexyl ester comprising reacting p-nitrobenzoic acid-2-ethylhexyl ester with hydrogen in the presence of a catalyst, wherein the reaction is performed
(a) in water; or
(b) in a mixture of water and 2-ethylhexanol.

It has been surprisingly found by the inventors, that performing the reaction in water or in a mixture of water and 2-ethylhexanol provides the desired product in high yield and purity, in particular without significant amounts of residual water. Performing the reaction in water or in a mixture of water and 2-ethylhexanol is advantageous with respect to the heat dissipation due to the beneficial specific heat capacity provided by water. This particularly applies as the preparation of p-aminobenzoic acid-2-ethylhexyl ester according to the invention is exothermic. Further, since water is formed as side product during the hydrogenation reaction, it is advantageous to also perform the reaction in water, which avoids additional contamination of the product and simplifies purification and solvent recycling. If the reaction is performed (a) in water, no additional solvent has to be removed at the end of the reaction. Further, no additional solvent is contaminated with water, which would have to be wasted or dewatered for recycling, reducing complexity, waste and costs. Since 2-ethylhexanol can be used as solvent in the preceding process step of forming the starting material p-nitrobenzoic acid-2-ethyl hexylester, also performing the reaction (b) in a mixture of water and 2-ethylhexanol is beneficial. In particular, the p-nitrobenzoic acid-2-ethyl hexylester can be applied directly dissolved in 2-ethylhexanol, which decreases complexity, waste and costs of the overall process. However, compared to performing the reaction in 2-ethylhexanol only, as in the prior art, the mixture with water provides advantageous specific heat capacity and thus advantageous heat dissipation.

Thus, the present invention provides an environmentally and economically advantageous process for the preparation of p-nitrobenzoic acid-2-ethyl hexylester in high purity and yield, which is suitable for large scale industrial applications.

In one embodiment of the invention, the reaction is performed in water.

In one embodiment of the invention, the catalyst is a precious metal, preferably palladium, Raney Nickel, rhodium, platinum or a mixture thereof.

In one embodiment of the invention, the reaction is performed at a temperature of from 70° C. to 150° C. for a time period of up to 24 h under up to 100 bar hydrogen pressure.

In one embodiment of the invention, after completion of the reaction, the reaction mixture is kept under hydrogen pressure for a ripening time of at least 60 min, preferably at least 90 min.

In one embodiment of the invention, the p-aminobenzoic acid-2-ethyl hexyl ester is purified by distillation.

In one embodiment of the invention, a stabilizer is added for the distillation wherein the stabilizer is selected from ascorbic acid, butylated hydroxytoluene (BHT), tocopherols such as vitamin E, carotenoids, or mixtures thereof.

In one embodiment of the invention, the process further comprises preparing 2,4,6-trianilino-p-(carbo-2′-ethylhexyl-1′-oxy)-1,3,5-triazine having the following chemical formula

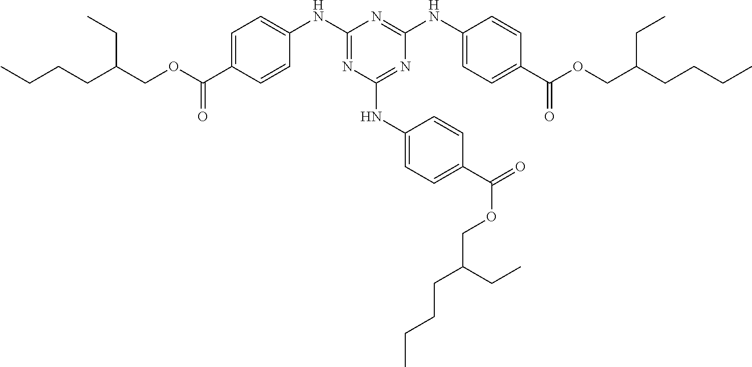 [SMILES: CCCCC(CC)COC(=O)c1ccc(Nc2nc(Nc3ccc(C(=O)OCC(CC)CCCC)cc3)nc(Nc3ccc(C(=O)OCC(CC)CCCC)cc3)n2)cc1]

by reacting a cyanuric halide with the p-aminobenzoic acid-2-ethyl hexylester in a non-polar solvent.

In one embodiment of the invention, the process further comprises preparing p-nitrobenzoic acid-2-ethyl hexylester comprising reacting p-nitrobenzoic acid with 2-ethylhexanol.

In one embodiment, the present invention relates to p-aminobenzoic acid-2-ethyl hexylester having a purity of at least 94% by weight.

In one embodiment, the present invention relates to p-aminobenzoic acid-2-ethyl hexylester, wherein the amount of bis(2-ethylhexyl)-4,4′-(diazene-1,2-diyl)(E)-dibenzoate having the following chemical formula

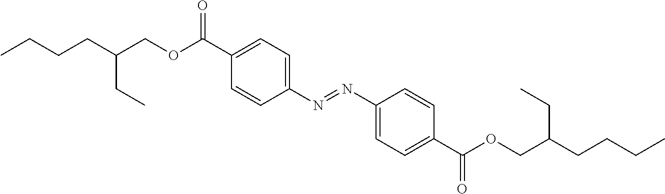 [SMILES: CCCCC(CC)COC(=O)c1ccc(/N=N/c2ccc(C(=O)OCC(CC)CCCC)cc2)cc1]

is less than 3000 ppm.

In one embodiment, the present invention relates to p-aminobenzoic acid-2-ethyl hexylester, which is obtained by the process of the present invention.

Preferred embodiments of the present invention can be found in the claims, the description and the examples. It is to be understood that the features mentioned above and those still to be illustrated below of the subject matter of the invention are preferred not only in the respective given combination but also in other combinations without leaving the scope of the invention.

In connection with the above embodiments of the present invention, the following definitions are provided.

In the context of the present invention, p-aminobenzoic acid-2-ethyl hexylester is obtained by a process comprising reacting p-nitrobenzoic acid-2-ethyl hexylester with hydrogen in the presence of a catalyst, wherein the reaction is performed (a) in water; or (b) in a mixture of water and 2-ethylhexanol.

As used herein, “p-aminobenzoic acid-2-ethyl hexylester” refers to para-aminobenzoic acid-2-ethyl hexylester, which represents 4-aminobenzoic acid-2-ethyl hexylester.

As used herein, “p-nitrobenzoic acid-2-ethyl hexylester” refers to para-nitrobenzoic acid-2-ethyl hexylester, which represents 4-nitrobenzoic acid-2-ethyl hexylester.

As used herein, the “catalyst” is a homogeneous or heterogeneous catalyst suitable for hydrogenation reactions. Exemplary catalysts are catalysts containing palladium, nickel, rhodium, platinum, iridium, ruthenium, iron or a mixture thereof. Preferably, the catalyst is a precious metal or a mixture thereof. More preferably, the catalyst is palladium, Raney Nickel, rhodium, platinum or a mixture thereof.

As used herein, “water” refers to deionized, demineralized, or distilled water, which refers to water that has been purified in such a way that (most of) its mineral- and salt ions are removed. As used herein, if the reaction is performed (a) in water or (b) in a mixture of water and 2-ethylhexanol, this refers to water being actively added to the reaction mixture at the beginning of the reaction and not to water only being present in the amount formed during the reaction. The process may thus comprise the step of adding water or the mixture of water and 2-ethylhexanol to the reactant, i.e. the p-nitrobenzoic acid-2-ethyl hexylester, or a reaction mixture comprising the reactant at the beginning of the reaction. The inventive process may thus comprise a first step of combining the p-nitrobenzoic acid-2-ethyl hexylester and, optionally, the catalyst with the solvent, i.e. the water or the water-2-ethylhexanol mixture before the reaction begins. The reaction may then be started by starting the hydrogen feed into the previously formed mixture.

In various embodiments, the present invention is thus directed to a process for preparing p-aminobenzoic acid-2-ethylhexyl ester by reacting p-nitrobenzoic acid-2-ethylhexyl ester with hydrogen in the presence of a catalyst, the process comprising providing a reaction mixture comprising p-nitrobenzoic acid-2-ethylhexyl ester and the catalyst in (a) water or (b) a mixture of water and 2-ethylhexanol; and carrying out the reaction of the p-nitrobenzoic acid-2-ethylhexyl ester with hydrogen in the presence of a catalyst in said water or in said mixture of water and 2-ethylhexanol.

Preferably, in (b) water is present in a ratio of at least 1:2 relative to 2-ethylhexanol, more preferably of at least 1:1 relative to 2-ethylhexanol and even more preferably of at least 2:1 relative to 2-ethylhexanol.

The ratios are preferably weight ratios, but in some embodiments may be volume ratios. Both options are thus covered by the given ratios of at least 2:1, at least 1:1 and at least 1:2. In a preferred mixture of (b), the amount of 2-ethylhexanol is the amount of 2-ethylhexanol, which the starting material p-nitrobenzoic acid-2-ethyl hexylester is dissolved in or which the starting material p-nitrobenzoic acid-2-ethyl hexylester is contaminated with and no 2-ethylhexanol is additionally added.

In some embodiments of the invention, after completion of the reaction, the reaction mixture is kept under hydrogen pressure for a ripening time. As used herein, “ripening time” refers to the time period after the reaction is completed, wherein the reaction mixture is kept under hydrogen pressure. Thus, the reaction is only performed for the time necessary for completion of the reaction. If a time period is given for the reaction, the time period only refers to the range of time, wherein the reaction is completed depending on the given conditions and the reaction is not performed longer than necessary for completion of the reaction even if the given time period includes longer reaction times. For example, if the reaction is performed for a time period of up to 24 h, the reaction is not performed for 24 h if the reaction is completed after 15 h; in this case the reaction is performed for 15 h. Afterwards, ripening time starts. As used herein, “kept under hydrogen pressure” refers to hydrogen not being completely released from the reaction vessel/reactor. This includes keeping a similar, reduced or increased hydrogen pressure as compared to the reaction. Preferably, the hydrogen pressure during the ripening time is similar to the hydrogen pressure during the reaction or refers to the hydrogen pressure that results in the vessel/reactor after closing the hydrogen valve. Reaction time ends and ripening time starts when at least one of the reactants is fully converted and/or when the amount of at least one of the reactants is no longer changing.

In particular, ripening time of the process of the invention starts when the p-nitrobenzoic acid-2-ethyl hexylester is fully converted or the amount of the p-nitrobenzoic acid-2-ethyl hexylester has reached a minimum, and/or the hydrogen consumption has reached a minimum. As used herein “has reached a minimum” refers to having reached an absolute minimum such that the amount of p-nitrobenzoic acid-2-ethyl hexylester or the hydrogen consumption are no longer changing, respectively. To determine completion of the reaction and thus start of the ripening time, the reaction can be monitored by methods known to the person skilled in the art, e.g., thin layer chromatography, GC, HPLC, or NMR. If p-nitrobenzoic acid-2-ethyl hexylester is no longer detected or a decrease in the amount of p-nitrobenzoic acid-2-ethyl hexylester is no longer detected, the reaction is completed, and the ripening time starts.

Further, completion of the reaction and thus start of the ripening time can be determined by monitoring the amount of hydrogen consumption, e.g., by monitoring the hydrogen flow while maintaining a constant hydrogen pressure. If the hydrogen flow while maintaining a constant hydrogen pressure has reached a minimum, the reaction is completed, and the ripening time starts. During ripening time, the reaction mixture is kept under hydrogen pressure. Thus, ripening time ends with the release of the remaining hydrogen from the reaction vessel/reactor.

In some embodiments, the product is purified by distillation, wherein a stabilizer is used. As used herein, “stabilizer” refers to one stabilizer or a mixture of stabilizers used to stabilize the product p-aminobenzoic acid-2-ethyl hexylester during distillation, e.g., by preventing polymerization and/or oxidation of the p-aminobenzoic acid-2-ethyl hexylester. Thus, a stabilizer can be an acid and/or base, such as a carboxylic acid and/or salt of a carboxylic acid to neutralize acids and/or bases present in the mixture, which induce polymerization of the p-aminobenzoic acid-2-ethyl hexylester. Further, the stabilizer can be an antioxidant. Exemplary stabilizers are ascorbic acid, butylated hydroxytoluene (BHT), tocopherols such as vitamin E, carotenoids, or mixtures thereof. A preferred stabilizer is ascorbic acid.

As used herein, the pressure indicated by the pressure unit “bar” refers to relative pressure or gauge pressure, which is zero-referenced against ambient air pressure, so it is equal to absolute pressure minus atmospheric pressure. Gauge pressure may also be indicated by the pressure unit “barg”. Only in case the pressure unit “bar(abs)” is used, the pressure refers to absolute pressure.

Preferred embodiments regarding the process of the invention are described hereinafter.

The following general considerations apply to the process.

In general, the reaction steps are performed in reaction vessels customary for such reactions, e.g., conventional stirred tank reactors. The reactions may be carried out in a continuous, semi-batch-wise or batch-wise manner. Preferably, the process of the invention is carried out in a batch-wise manner, wherein all reactants are provided in the reaction vessel before starting the reaction.

Details regarding the reaction pressure and temperature are provided below. If not indicated otherwise, the process steps are preferably carried out under atmospheric pressure. The end of the reaction can be monitored by methods known to a person skilled in the art, e.g., thin layer chromatography, GC, HPLC or NMR.

If not otherwise indicated, the reactants can in principle be contacted with one another in any desired sequence.

Furthermore, it is emphasized that the reaction may be performed on a laboratory scale and industrial scale.

In the following, preferred embodiments of the invention are provided. It is to be understood that the preferred embodiments of the invention are preferred alone or in combination with each other.

As indicated above, the present invention relates to a process for preparing p-aminobenzoic acid-2-ethyl hexylester comprising reacting p-nitrobenzoic acid-2-ethyl hexylester with hydrogen in the presence of a catalyst, wherein the reaction is performed (a) in water, or (b) in a mixture of water and 2-ethylhexanol. If the reaction is performed in water or in a mixture of water and 2-ethylhexanol, water provides advantageous specific heat capacity and thus advantageous heat dissipation of the reaction mixture, which is particularly relevant for the exothermic preparation of p-aminobenzoic acid-2-ethyl hexylester.

Further, since water is also formed as side product during the reaction, the number of contaminants in the mixture is not increased by adding water to the reaction mixture. If the reaction is performed (a) in water, no solvent contaminants other than water have to be removed. As water has to be removed in any case as it forms as side product during the reaction, the process of the present invention decreases the complexity of product purification and recycling and thus the waste and costs of the process. Thus, in one embodiment of the invention, the reaction is performed in water. It is also possible to apply the starting material p-nitrobenzoic acid-2-ethyl hexylester dissolved in 2-ethylhexanol or contaminated with 2-ethylhexanol. This is particularly advantageous since the preceding step of preparing p-nitrobenzoic acid-2-ethyl hexylester can be performed in 2-ethylhexanol such that the step of solvent removal prior to the reaction of the present invention can be avoided or reduced with positive effects on the complexity, waste, and costs of the overall process of preparing p-aminobenzoic acid-2-ethyl hexylester. Also in this case, adding water as solvent provides advantageous specific heat capacity and thus advantageous heat dissipation of the reaction mixture. Thus, in one embodiment of the invention, the reaction is performed in a mixture of water and 2-ethylhexanol. In one embodiment, the reaction is performed in a mixture of water and 2-ethylhexanol with a weight ratio of water to 2-ethylhexanol of at least 1:2. In another embodiment, the reaction is performed in a mixture of water and 2-ethylhexanol with a weight ratio of water to 2-ethylhexanol of at least 1:1. In another embodiment, the reaction is performed in a mixture of water and 2-ethylhexanol with a weight ratio of water to 2-ethylhexanol of at least 2:1. Increasing the amount of water relative to the amount of 2-ethylhexanol increases the specific heat capacity of the reaction mixture with a positive effect on the heat dissipation. Thus, in a preferred embodiment, the amount of 2-ethylhexanol in the mixture of water and 2-ethylhexanol is the amount of 2-ethylhexanol, which the starting material p-nitrobenzoic acid-2-ethyl hexylester is dissolved in or which the starting material p-nitrobenzoic acid-2-ethyl hexylester is contaminated with and no 2-ethylhexanol is additionally added.

Further, in one embodiment of the invention, the pH value is adjusted to a value in the range of from 4 to 10 prior to the reaction with hydrogen. In another embodiment, the pH value is adjusted to neutral values. In one embodiment, the pH value is adjusted using sodium hydroxide. By adjusting the pH prior to the reaction with hydrogen, the purity of the p-aminobenzoic acid-2-ethyl hexylester can be increased.

In one embodiment of the invention, the catalyst is a precious metal. Preferably, the catalyst is selected from the group consisting of palladium, Raney Nickel, rhodium, platinum and a mixture thereof. In one embodiment, the catalyst is palladium. In another embodiment, the catalyst is palladium on charcoal (Pd/C). In another embodiment, the catalyst is palladium on charcoal (Pd/C), wherein the amount of palladium is in the range of from 1 weight-% to 10 weight-% palladium. In another embodiment, the catalyst is 2 weight-% palladium on charcoal (Pd/C). The amount of catalyst refers to the amount of precious metal relative to the amount of p-nitrobenzoic acid-2-ethyl hexylester. In one embodiment, 0.002-0.005 weight-% precious metal relative to p-nitrobenzoic acid-2-ethyl hexylester are used. In another embodiment, 0.003-0.004 weight-% precious metal relative to p-nitrobenzoic acid-2-ethyl hexylester are used.

The reaction is performed for the time period until the reaction is completed. Afterwards, ripening time starts. Thus, depending on the hydrogen pressure and the temperature applied, the reaction time can vary. For example, the reaction time can be shorter by performing the reaction under higher hydrogen pressures. To determine completion of the reaction, the reaction can be monitored by methods known to the person skilled in the art, e.g., thin layer chromatography, GC, HPLC, or NMR. If p-nitrobenzoic acid-2-ethyl hexylester is no longer detected or a decrease in the amount of p-nitrobenzoic acid-2-ethyl hexylester is no longer detected, the reaction is completed. Further, completion of the reaction can be determined by monitoring the amount of hydrogen consumption, e.g., by monitoring the hydrogen flow while maintaining a constant hydrogen pressure. If the hydrogen flow while maintaining a constant hydrogen pressure has reached a minimum, the reaction is completed. In one embodiment of the invention, the reaction is performed at a temperature of from 70° C. to 150° C. for a time period of up to 24 h under up to 100 bar hydrogen pressure. In another embodiment, the reaction is performed at a temperature of from 70° C. to 150° C. for a time period of up to 24 h under a hydrogen pressure of from 1 to 50 bar. In another embodiment, the reaction is performed at a temperature of from 70° C. to 150° C. for a time period of up to 24 h under a hydrogen pressure of from 2 to 20 bar. In another embodiment, the reaction is performed at a temperature of from 70° C. to 150° C. for a time period of up to 24 h under a hydrogen pressure of from 7 to 10 bar.

In one embodiment of the invention, the reaction is performed at a temperature of from 80° C. to 120° C. for a time period of up to 24 h under up to 100 bar hydrogen pressure. In another embodiment, the reaction is performed at a temperature of from 80° C. to 120° C. for a time period of up to 24 h under a hydrogen pressure of from 1 to 50 bar. In another embodiment, the reaction is performed at a temperature of from 80° C. to 120° C. for a time period of up to 24 h under a hydrogen pressure of from 2 to 20 bar. In another embodiment, the reaction is performed at a temperature of from 80° C. to 120° C. for a time period of up to 24 h under a hydrogen pressure of from 7 to 10 bar.

In one embodiment of the invention, the reaction is performed at a temperature of from 90° C. to 100° C. for a time period of up to 24 h under up to 100 bar hydrogen pressure. In another embodiment, the reaction is performed at a temperature of from 90° C. to 100° C. for a time period of up to 24 h under a hydrogen pressure of from 1 to 50 bar. In another embodiment, the reaction is performed at a temperature of from 90° C. to 100° C. for a time period of up to 24 h under a hydrogen pressure of from 2 to 20 bar. In another embodiment, the reaction is performed at a temperature of from 90° C. to 100° C. for a time period of up to 24 h under a hydrogen pressure of from 7 to 10 bar. In one particular embodiment of the invention, the reaction is performed at a temperature of from 90° C. to 100° C. for a time period of from 10 h to 20 h under a hydrogen pressure of from 2 to 20 bar. In another particular embodiment, the reaction is performed at a temperature of from 90° C. to 100° C. for a time period of from 13 h to 17 h under a hydrogen pressure of from 7 to 10 bar. As described above, the reaction is not performed longer than necessary for completion of the reaction. If a time period is given for the reaction, the time period only refers to the range of time, wherein the reaction is completed depending on the given conditions and the reaction is not performed longer than necessary for completion of the reaction even if the given time period includes longer reaction times. For example, if the reaction is performed for a time period of up to 24 h, the reaction is not performed for 24 h if the reaction is completed after 15 h; in this case the reaction is performed for 15 h. Afterwards, ripening time starts.

It has been surprisingly found by the inventors, that providing a ripening time, wherein the reaction mixture is kept under hydrogen pressure after completion of the reaction, increases the purity of the p-aminobenzoic acid-2-ethyl hexylester. In particular, contamination with the azo-compound bis(2-ethylhexyl)-4,4′-(diazene-1,2-diyl)(E)-dibenzoate having the following chemical formula

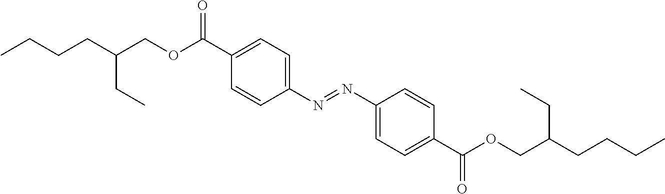 [SMILES: CCCCC(CC)COC(=O)c1ccc(/N=N/c2ccc(C(=O)OCC(CC)CCCC)cc2)cc1]

as side product is significantly reduced or even avoided.

Thus, in one embodiment of the invention, after completion of the reaction, the reaction mixture is kept under hydrogen pressure for a ripening time of at least 60 min, preferably at least 90 min. In another embodiment, after completion of the reaction, the reaction mixture is kept under hydrogen pressure for a ripening time of at least 2 h. In another embodiment, after completion of the reaction, the reaction mixture is kept under hydrogen pressure for a ripening time of at least 3 h. In another embodiment, after completion of the reaction, the reaction mixture is kept under hydrogen pressure for a ripening time of from 2 h to 4 h. In another embodiment, after completion of the reaction, the reaction mixture is kept under hydrogen pressure for a ripening time of from 2.5 to 3.5 h. Extending the ripening time does not negatively affect the reduction or avoidance of the formation of the azo-compound, but decreases the space-time-yield, i.e., the yield obtained per space and time in the reactor/vessel, which increases costs. In one embodiment, the hydrogen pressure during the ripening time is from 2 bar to 20 bar. In another embodiment, the hydrogen pressure during the ripening time is from 7 to 10 bar. In one embodiment, the hydrogen pressure during the ripening time is similar to the hydrogen pressure during the reaction or the hydrogen pressure resulting in the reactor/vessel after closing of the hydrogen valve. In another embodiment, the hydrogen pressure during ripening time is similar to the hydrogen pressure during the reaction for 1.5 to 2 h, followed by the hydrogen pressure resulting in the reactor/vessel after closing of the hydrogen valve for 0.5 to 1.5 h. In one embodiment, the temperature during the ripening time is from 70° C. to 150° C. In another embodiment, the temperature during the ripening time is from 80° C. to 120° C.

In another embodiment, the temperature during the ripening time is from 90° C. to 100° C. In one embodiment, the temperature during the ripening time is similar to the reaction temperature. In another embodiment, the temperature during the ripening time is similar to the reaction temperature and the hydrogen pressure during the ripening time is similar to the hydrogen pressure during the reaction for 1.5 to 2 h, followed by the hydrogen pressure resulting in the reactor/vessel after closing of the hydrogen valve for 0.5 to 1.5 h. In another embodiment, the temperature during the ripening time is from 70° C. to 100° C. and the hydrogen pressure during the ripening time is from 2 bar to 20 bar for 1.5 to 2 h, followed by the hydrogen pressure resulting in the reactor/vessel after closing of the hydrogen valve for 0.5 to 1.5 h.

To further purify the p-aminobenzoic acid-2-ethyl hexylester, the solvent and optionally side products of the reaction can be removed by distillation. Thus, in one embodiment of the invention, the p-aminobenzoic acid-2-ethyl hexylester is purified by distillation. The solvent removed by distillation can be recycled. In particular, the solvent can be re-used in the next process of reducing p-nitrobenzoic acid-2-ethyl hexylester to p-aminobenzoic acid-2-ethyl hexylester. Further, in one embodiment of the invention, a stabilizer is added for the distillation. In particular, the stabilizer can be one compound or more than one compound. In one embodiment, the stabilizer is one compound. Adding a stabilizer increases the yield and purity of the p-aminobenzoic acid-2-ethyl hexylester, in particular by preventing polymerization and/or oxidation of the p-aminobenzoic acid-2-ethyl hexylester. Thus, in one embodiment, the stabilizer is a carboxylic acid and/or a salt of a carboxylic acid to neutralize acids and/or bases present in the mixture, which induce polymerization of the p-aminobenzoic acid-2-ethyl hexylester. In another embodiment, the stabilizer is an antioxidant. In another embodiment, the stabilizer is an antioxidant and a carboxylic acid and/or a salt of a carboxylic acid. In one embodiment, the stabilizer is selected from the group consisting of ascorbic acid, butylated hydroxytoluene (BHT), tocopherols, or mixtures thereof. In one embodiment, the stabilizer is ascorbic acid or a mixture with ascorbic acid, preferably the stabilizer is ascorbic acid.

In one embodiment of the invention, after the reaction with hydrogen, the reaction mixture is filtered to remove the solid catalyst and phase separation of the filtrate is performed. In one embodiment, the catalyst is removed with a bag filter. In one embodiment, phase separation is performed by adding a carbonate salt. Performing phase separation by adding a carbonate salt further reduces the water content in the organic phase. Thereby, the purity of the p-aminobenzoic acid-2-ethylhexylester in the organic phase is increased. Further, removing water from the p-aminobenzoic acid-2-ethyl hexylester product is particularly advantageous for the subsequent reaction of p-aminobenzoic acid-2-ethyl hexylester with a cyanuric halide to ethylhexyl triazone since cyanuric halides have the tendency to hydrolyze giving rise to mono-, di-, and/or trihydroxy-triazines, which would have to be removed by additional process steps.

As indicated above, the process of the present invention provides a process for preparing p-aminobenzoic acid-2-ethyl hexylester, which is an important precursor for the preparation of the known highly effective UV absorber ethylhexyl triazone (Uvinul T 150, CAS number: 88122-99-0). Said ethylhexyl triazone represents 2,4,6-trianilino-p-(carbo-2′-ethylhexyl-1′-oxy)-1,3,5-triazine. Thus, in another embodiment, the process further comprises preparing 2,4,6-trianilino-p-(carbo-2′-ethylhexyl-1′-oxy)-1,3,5-triazine having the following chemical formula

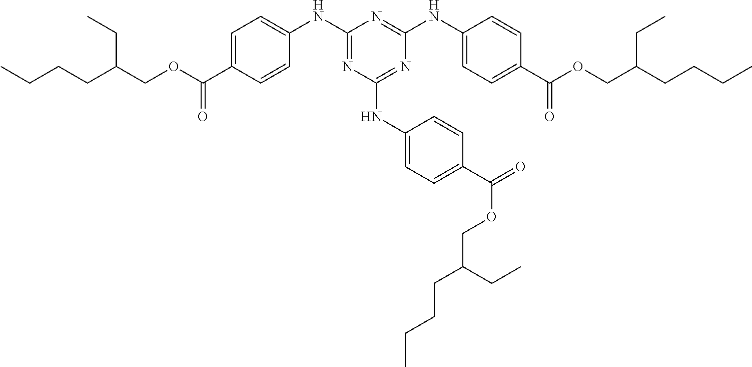 [SMILES: CCCCC(CC)COC(=O)c1ccc(Nc2nc(Nc3ccc(C(=O)OCC(CC)CCCC)cc3)nc(Nc3ccc(C(=O)OCC(CC)CCCC)cc3)n2)cc1]

by reacting a cyanuric halide with the p-aminobenzoic acid-2-ethyl hexylester in a non-polar solvent. Preferably, the cyanuric halide is reacted with the p-aminobenzoic acid-2-ethyl hexylester in a molar ratio of from 1:3 to 1:5. Reacting the cyanuric halide with the p-aminobenzoic acid-2-ethyl hexylester in a molar ratio of at least 1:3 allows for full conversion of the cyanuric halide since 3 equivalents of p-aminobenzoic acid-2-ethyl hexylester relative to 1 equivalent of cyanuric halide are needed to form ethylhexyl triazone. The improved process for preparing p-aminobenzoic acid-2-ethyl hexylester is therefore also advantageous for the preparation of ethylhexyl triazone. Improving the purity of p-aminobenzoic acid-2-ethyl hexylester while reducing complexity, waste, and cost of its preparation also reduces the overall complexity, waste, and cost of the preparation of ethylhexyl triazone. This is particularly advantageous considering the high demand for ethylhexyl triazone due to its various applications as UV absorber.

Further, the p-aminobenzoic acid-2-ethyl hexylester is prepared by reacting p-nitrobenzoic acid-2-ethyl hexylester with hydrogen. Thus, in one embodiment of the invention, the process further comprises preparing p-nitrobenzoic acid-2-ethyl hexylester comprising reacting p-nitrobenzoic acid with 2-ethylhexanol. In one embodiment, the p-nitrobenzoic acid-2-ethyl hexylester prepared by this process is directly employed in the subsequent reaction to prepare the p-aminobenzoic acid-2-ethyl hexylester without purification or removal of the solvent. This particularly applies if the preparation of the p-nitrobenzoic acid-2-ethyl hexylester is performed in water or 2-ethylhexanol or mixtures thereof, or without any solvent.

As described above, high purity can be obtained by the process of this invention. Thus, in one embodiment, the present invention relates to a p-nitrobenzoic acid-2-ethyl hexylester having a purity of at least 94% by weight or at least 94% as determined by GC (AUC). The purity may be higher, for example at least 95% or at least 96%, or even at least 97% or 98%. In particular, by keeping the reaction mixture under hydrogen pressure for a ripening time after completion of the reaction significantly reduces or even avoids contamination with the azo compound bis(2-ethylhexyl)-4,4′-(diazene-1,2-diyl)(E)-dibenzoate. Thus, in one embodiment, the present invention relates to a p-aminobenzoic acid-2-ethyl hexylester, wherein the amount of bis(2-ethylhexyl)-4,4′-(diazene-1,2-diyl)(E)-dibenzoate having the following chemical formula

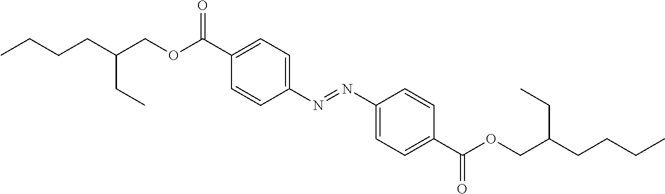 [SMILES: CCCCC(CC)COC(=O)c1ccc(/N=N/c2ccc(C(=O)OCC(CC)CCCC)cc2)cc1]

is less than 3000 ppm, for example less than 2500 ppm or less than 2000 ppm.

Further, in one embodiment, the present invention relates to a p-nitrobenzoic acid-2-ethyl hexylester, which is obtained by the process of this invention. This may be characterized by the low amounts of the azo compound bis(2-ethylhexyl)-4,4′-(diazene-1,2-diyl)(E)-dibenzoate, as described above.

The present invention is further illustrated by the following examples.

### EXAMPLES

In the examples, GC analysis is performed on an Agilent Technologies 6890N using the following conditions and parameters:
Detector: FID
Solvent: Acetonitrile
Column: Optima 5 (30 m, 0.32 mm ID, 0.25 μm film thickness)
Carrier Gas: nitrogen

### Example 1: Preparation of p-Aminobenzoic Acid-2-Ethylhexyl Ester without Ripening

8 kg of p-nitrobenzoic acid-2-ethylhexyl ester is added to the reactor with a pre-charge of 0.75 L of deionized water under stirring. The pH-value of the mixture is adjusted to neutral values using sodium hydroxide (50 weight-% in water). 0.015 kg of catalyst (2 weight-% palladium on charcoal) is added under stirring to the reactor. The temperature is adjusted to 95° C. and hydrogen at 9 barg is introduced under vigorous stirring. The hydrogen flow is closely monitored throughout, while maintaining a pressure of 9 barg. The reaction is stopped if the hydrogen flow is reaching a minimum. Afterwards the residual hydrogen is released. The residual catalyst is removed from the reaction mixture using a bag filter. The stirrer is stopped to allow the organic phase containing the product to separate from the water phase. The lower phase consisting of water is then removed from the bottom of the reactor. The organic phase containing the product p-aminobenzoic acid-2-ethylhexyl ester remains in the reactor and is subsequently analyzed. The yield was 7.14 kg organic phase. Characterization of the final product was done using GC and the water content was measured using Karl-Fischer titration. The results are shown in Table 1.

TABLE 1

| | | |
|---|---|---|
| Water content | 2.0 | weight-% |
| Azo aminoester | 9.4 | area-% |
| p-aminobenzoic acid-2-ethylhexyl ester | 85.0 | area-% |
| p-Nitrobenzoic acid-2-ethylhexyl ester | 0.1 | area-% |

### Example 2: Preparation of p-Aminobenzoic Acid-2-Ethylhexyl Ester with Ripening

8 kg of p-nitrobenzoic acid-2-ethylhexyl ester is added to the reactor with a pre-charge of 0.75 L of deionized water under stirring. The pH-value of the mixture is adjusted to neutral values using sodium hydroxide (50 weight-% in water).

0.015 kg of catalyst (2 weight-% palladium on charcoal) is added under stirring to the reactor.

The temperature is adjusted to 95° C. and hydrogen at 9 barg is introduced under vigorous stirring. The hydrogen flow is closely monitored throughout, while maintaining a pressure of 9 barg. The hydrogen pressure is held up for another 120 minutes once the hydrogen flow is reaching a minimum.

Subsequently the hydrogen valve is closed, and ripening was continued for another 60 minutes.

Afterwards the residual hydrogen is released.

The residual catalyst is removed from the reaction mixture using a bag filter.

The stirrer is stopped to allow the organic phase containing the product to separate from the water phase.

The lower phase consisting of water is then removed from the bottom of the reactor. The organic phase containing the product p-aminobenzoic acid-2-ethylhexyl ester remains in the reactor and is subsequently analyzed. The yield was 7.14 kg organic phase. Characterization of the final product was done using GC and the water content was measured using Karl-Fischer titration. The results are shown in Table 2.

TABLE 2

| | | |
|---|---|---|
| Water content | 2.2 | weight-% |
| Azo aminoester | 0.2 | area-% |
| p-aminobenzoic acid-2-ethylhexyl ester | 94.9 | area-% |
| p-Nitrobenzoic acid-2-ethylhexyl ester | 0.1 | area-% |

1.-12. (canceled)

13. A process for preparing p-aminobenzoic acid-2-ethyl hexylester comprising reacting p-nitrobenzoic acid-2-ethyl hexylester with hydrogen in the presence of a catalyst, wherein the reaction is performed
(a) in water; or
(b) in a mixture of water and 2-ethylhexanol
wherein the process comprises adding the water (a) or the mixture of water and 2-ethylhexanol to the reaction mixture at the beginning of the reaction.

14. The process according to claim 13, wherein the reaction is performed in water and wherein the process comprises adding said water to the reaction mixture at the beginning of the reaction.

15. The process according to claim 13, wherein the reaction is performed in a mixture of water and 2-ethylhexanol, wherein the ratio of water to 2-ethylhexanol is at least 1:2, and
wherein the process comprises adding said mixture of water and 2-ethylhexanol to the reaction mixture at the beginning of the reaction.

16. The process according to claim 13, wherein the catalyst is a precious metal.

17. The process according to claim 16, wherein the precious metal is palladium, Raney Nickel, rhodium, platinum or a mixture thereof.

18. The process according to claim 13, wherein the reaction is performed at a temperature of from 70° C. to 150° C. for a time period of up to 24 h under up to 100 bar hydrogen pressure.

19. The process according to claim 13, wherein after completion of the reaction, the reaction mixture is kept under hydrogen pressure for a ripening time of at least 60 min.

20. The process according to claim 13, wherein the p-aminobenzoic acid-2-ethyl hexyl ester is purified by distillation.

21. The process according to claim 19, wherein a stabilizer is added for the distillation, wherein the stabilizer is selected from ascorbic acid, butylated hydroxytoluene (BHT), tocopherols such as vitamin E, carotenoids, or mixtures thereof.

22. The process according to claim 13, wherein the process further comprises preparing 2,4,6-trianilino-p-(carbo-2′-ethylhexyl-1′-oxy)-1,3,5-triazine having the following chemical formula 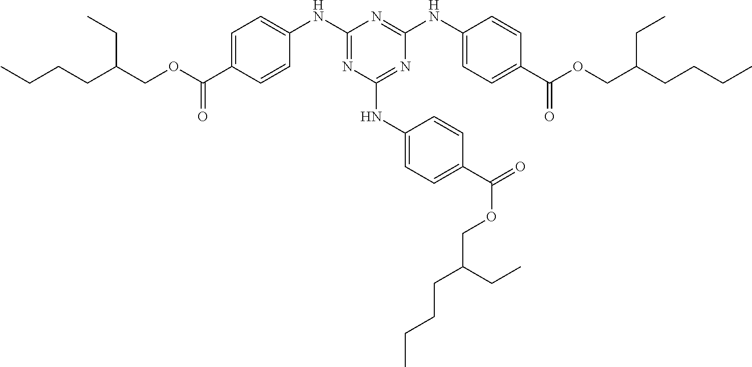 [SMILES: CCCCC(CC)COC(=O)c1ccc(Nc2nc(Nc3ccc(C(=O)OCC(CC)CCCC)cc3)nc(Nc3ccc(C(=O)OCC(CC)CCCC)cc3)n2)cc1]
by reacting a cyanuric halide with the p-aminobenzoic acid-2-ethyl hexylester in a non-polar solvent.

23. The process according to claim 13, wherein the process further comprises preparing p-nitrobenzoic acid-2-ethyl hexylester comprising reacting p-nitrobenzoic acid with 2-ethylhexanol.

24. A P-Aminobenzoic acid-2-ethyl hexylester having a purity of at least 94% by weight or 94% as determined by GC, wherein the amount of bis(2-ethylhexyl)-4,4′-(diazene-1,2-diyl)(E)-dibenzoate having the following chemical formula 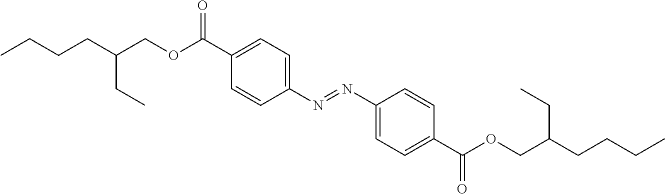 [SMILES: CCCCC(CC)COC(=O)c1ccc(/N=N/c2ccc(C(=O)OCC(CC)CCCC)cc2)cc1]
is less than 3000 ppm.

25. A P-Aminobenzoic acid-2-ethyl hexylester having a purity of at least 94% by weight or 94% as determined by GC, wherein the amount of bis(2-ethylhexyl)-4,4′-(diazene-1,2-diyl)(E)-dibenzoate having the following chemical formula 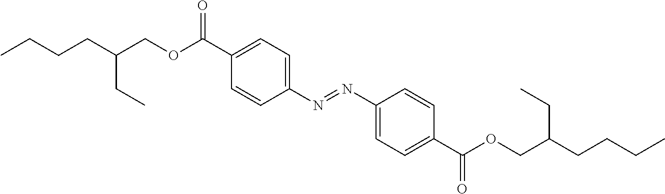 [SMILES: CCCCC(CC)COC(=O)c1ccc(/N=N/c2ccc(C(=O)OCC(CC)CCCC)cc2)cc1]
is less than 3000 ppm, which is obtained by the process of claim 13, wherein in said process after completion of the reaction, the reaction mixture is kept under hydrogen pressure for a ripening time of at least 60 min.
